(19) United States
(12) Patent Application Publication
(10) Pub. No.: US 2007/0066561 A1
(43) Pub. Date: Mar. 22, 2007

(54) Drug delivery and targeting with vitamin B12 conjugates

(75) Inventors: Stephen Wilson, Houston, TX (US); Kathryn Suzanne Reinhard, Garden City, NY (US); Xiang Gao, New York, NY (US)

Correspondence Address: BROWDY AND NEIMARK, P.L.L.C.;624 NINTH STREET, NW, SUITE 300, WASHINGTON, DC, 20001-5303, US

(73) Assignee: New York University, New York, NY (US)

(21) Appl. No.: 11/601,809

(22) Filed: Nov. 20, 2006

Related U.S. Application Data
(62) Division of application No. 10/235,857, filed on Sep. 6, 2002.

(51) Int. Cl. A61K 31/714

(52) U.S. Cl. 514/52

(57) ABSTRACT

Vitamin B₁₂ can be conjugated to anti-cancer drugs to deliver these drugs selectively to tumors, wherein the conjugates bind to transcobalamin. The complex of vitamin B₁₂ and transcobalamin is recognized and taken into cells by specific cell surface receptors which are overexpressed in rapidly dividing cells.

### CROSS-REFERENCE TO RELATED APPLICATIONS

The present application is a division of application U.S. Ser. No. 10/235,857, filed Sep. 6, 2002, which claims the benefit under 35 U.S.C. 119(e) of U.S. provisional application No. 60/314,189, filed Aug. 23, 2001, the entire contents of each of which are hereby incorporated by reference.

### BACKGROUND OF THE INVENTION

Vitamin B₁₂, also known as cyanocobalamin, belongs to the family of compounds known as the corrinoids. This family of compounds contain a common corrin nucleus, a partially hydrogenated tetrapyrrole in which two pyrroles are joined directly rather than by methane bridges. These compound possess a central cobalt atom bound by coordinate linkages to the nitrogen atoms of the four pyrroles.

Vitamin B₁₂ is further characterized by a distinct number of methyl, propionamide, and acetamide side chains attached to the pyrroles, and one side chain on the ring D in which the propionic acid is amidated with 1-amino-2-propanol. The latter is esterified with α-D-ribofuranosyl-(5,6-dimethylbenzimidazole)-3-phosphate. The fifth coordinate linkage with the cobalt atom in the α position is formed by the second N-atom of the 5,6-dimethylbenzimidazole. The β position is occupied by CN⁻ in cyanocobalamin, OH⁻ in hydroxycobalamin, H₂O in aquacobalamin, CH₃ in methylcobalamin, and deoxyadenosyl in adenosylcobalamin (coenzyme B₁₂).

Cyanocobalamin is moderately soluble in water at room temperature (12 g/liter) as well as in lower alcohols and phenol. However, it is insoluble in acetone, ether, and chloroform. Vitamin B₁₂ is neutral in water and quite stable in aqueous solutions between pH about 4 and 7. The molecular weight of vitamin B₁₂ is 1355.4, and the empirical formula is C₆₃H₈₈N₁₄O₁₄PCo. Although it is photosensitive, cyanocobalamin can be heated to about 120 EC without significant decomposition.

Vitamin B₁₂ is an essential co-factor for the biosynthesis of methionine and nucleic acids, and is imported into dividing cells via a receptor-mediated pathway. This receptor is overexpressed from about 100 to 1000 times in rapidly dividing cells such as tumor cells. Vitamin B₁₂ is a limiting component in the production of folates, and an essential carbon source for the synthesis of DNA. Once absorbed into the body, Vitamin B₁₂ is transported to the blood stream, where it is complexed with its carrier protein transcobalamin II (TCII). The B₁₂/TCII complex is recognized and taken into cells by specific cell surface receptors which are overexpressed in rapidly dividing cells.

Several studies have been reported in which vitamin B₁₂ conjugated to a variety of drugs was delivered to cancer patients. Collins et al., in Mayo Clin. Proc. 2000:75:568-580,describe a study of the biodistribution of a vitamin B₁₂ analog, indium In-111-labelled diethylenetriaminepentaacetate adenosylcobalamin in patients recently diagnosed as having primary or recurrent malignancy. It was found in this study that vitamin B₁₂ may be a useful vehicle for delivering diagnostic and therapeutic agents to various malignancies.

The Salt Lake Tribune on Thursday, Apr. 1, 1999, reported that some scientists at the University of Utah linked anticancer drugs to vitamin B₁₂, creating bioconjugates to carry chemotherapy drugs into tumor cells. However, this work was only conducted in vitro, and there is no indication that it would work in vivo.

Grissom et al., in WO9808859, disclose bioconjugates and delivery of bioactive agents targeted for site-specific release in cells, tissues or organs. The bioconjugates comprise a bioactive agent and an organocobalt complex to which the bioactive agent is covalently bonded directly or indirectly to the cobalt atom of the organocobalt complex. The bioactive agent is released from the bioconjugate by the cleavage of the covalent bond between the bioactive agent and the cobalt atom in the organocobalt complex. This cleavage may occur as a result of normal displacement by cellular nucleophiles or enzymatic action, but is preferably effected selectively at a predetermined release site by application of an external signal such as light, photoexcitation, or ultrasound. If the photolysis occurs in the presence of a magnetic field surrounding the release site, the release of the bioactive agent into surrounding healthy tissue is minimized.

Soda et al., in Blood 65(4): 1985, pp. 795-802 report on the receptor distribution and the endothelial uptake of transcobalamin II in liver cell suspensions. Visual probes were designed in which TCII was covalently coupled to submicrometer amide-modified latex particles. It was found that the binding of TCII minibeads was limited to endothelial cells.

Mitchell et al., in Enzymatic Mechanisms, P. A. Frey and D. B. Northrop, Eds., IOS Press, 1999, describe the use of the vitamin B₁₂ transport and receptor system to target the delivery of alkylating agents to leukemia cells. Leukemia cells internalized the chlorambucil-cobalamin bioconjugate through receptor-mediated endocytosis and appeared to have a higher requirement for cobalamin than non-transformed cells. Once the drug-cobalamin bioconjugate is internalized, leukemia cells cleave the Co—C bond, thereby separating the drug from the cobalamin carrier and releasing the active alkylating agent inside the cell.

Trakahashi et al., in Nature 288, 713-715 (1980) note that membrane transport of vitamin B₁₂ into mammalian cells is mediated by the serum protein transcobalamin II. When L1210 cells were incubated with minibeads containing TCII-cobalamin and examined by scanning electron microscopy, the particles were found attached predominantly to microvilli. Incubation of the cells resulted in the internalization of the minibeads.

One goal of modern pharmacology has been to refine a targeted drug delivery system via a magic bullet that seeks cancer cells while sparing healthy cells. However, only partial selectivity has been achieved with traditional drugs, polymers, liposomes, and monoclonal antibodies. The reason for this is that either the drug reaches all of the target cells but has an undesirable affinity for healthy cells, or it does not act on any of the non-targeted cells, but reaches only some of the malignant cells.

### SUMMARY OF THE INVENTION

It is an object of the present invention to overcome the aforementioned deficiencies in the prior art.

It is another object of the present invention to provide bioconjugates for delivery of drugs to predetermined locations for chemotherapy.

It is another object of the present invention to provide improved selectivity in targeting drugs.

It is a further object of the present invention to use vitamin B₁₂ as a vehicle to deliver drugs selectively to tumors.

According to the present invention, vitamin B₁₂ is conjugated to a drug via a linker group. This conjugate provides an antitumor effect as a result of a specific, receptor-mediated event, and only works in the presence of the vitamin B₁₂ carrier protein TCII, which is required for cell surface receptor binding and subsequent internalization.

The present invention is based on the fact that vitamin B₁₂ is an essential co-factor for the biosynthesis of methionine and nucleic acids and is imported into dividing cells via a receptor-mediated pathway. This receptor is overexpressed, from about 100 to about 1000 times, in rapidly dividing cells such as tumor cells. The structure of vitamin B₁₂ is shown in FIG. 1. This compound includes six primary amides around the central corrin ring structures, which primary amides are labeled, a-e and g. It was found that conjugation at the e-position proves most useful in drug delivery because conjugates at the e-position bound most strongly with TCII, which is required for the receptor-mediated drug delivery of the present invention.

The linkers can be any linkers that covalently bond vitamin B₁₂ to a drug. However, there should be a suitable spacing between the vitamin B₁₂ and the drug so that the drug is sufficiently exposed to the cell. Additionally, the spacer may be chosen to enhance the overall water solubility of the bioconjugates.

The linkers, or spacers, can include any linkers including B(CH₂)ₙ, wherein n is from 4 to 20. However, it has been found that longer chain linkers are preferred, i.e., where n>10. To increase the water solubility of the conjugate, linkers including ether groups, i.e., (CH₂CH₂O)n, where n is from 2-4, are the best.

The conjugates provide a much better toxicity profile than the unconjugated drugs because the B₁₂-toxin/drug is selectively taken up by cancer cells 10-1000 fold better than the unconjugated drug/toxin, so that much lower dosages of the drug/toxin are required. Also, because the uptake is TCII-mediated, toxicity can be prevented by dosing with plain B₁₂, which ties up the TCII and reverses or blocks further uptake.

### BRIEF DESCRIPTION OF THE DRAWINGS

FIG. 1 shows the structure of vitamin B₁₂ with the six primary amides labeled.

FIG. 2 shows binding curves for cyanocobalamin and analogs with rh TCII.

FIG. 3 illustrates synthesis of 4-iodohippuryl-4,7,10-trioxa-1,13-tridecanediamine-e-carboxylate analog.

FIG. 4 shows the formulae for three known anti-cancer drugs: taxol, doxorubicin, and cisplatin.

FIG. 5 shows the synthesis of vitamin B₁₂-taxol conjugate.

FIG. 6 shows the synthesis of vitamin B₁₂-dotorubicin conjugate.

FIG. 7 shows the synthesis of vitamin B₁₂-cisplatin conjugate.

FIG. 8 shows the effect of a vitamin B₁₂-taxol conjugate according to the present invention on the leukemia lymphoma cell line P388 in mice.

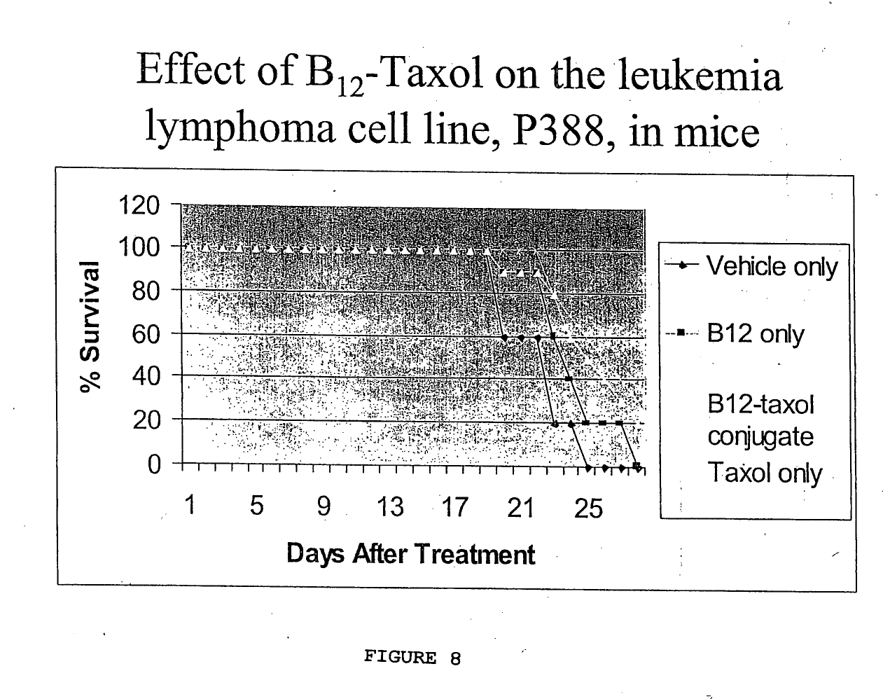

FIG. 9 shows % survival of mice after treatment with a B₁₂-taxol conjugate B₁₂ according to the present invention.

FIG. 10 illustrates the reaction scheme for preparing a vitamin B₁₂-cisplatin conjugate.

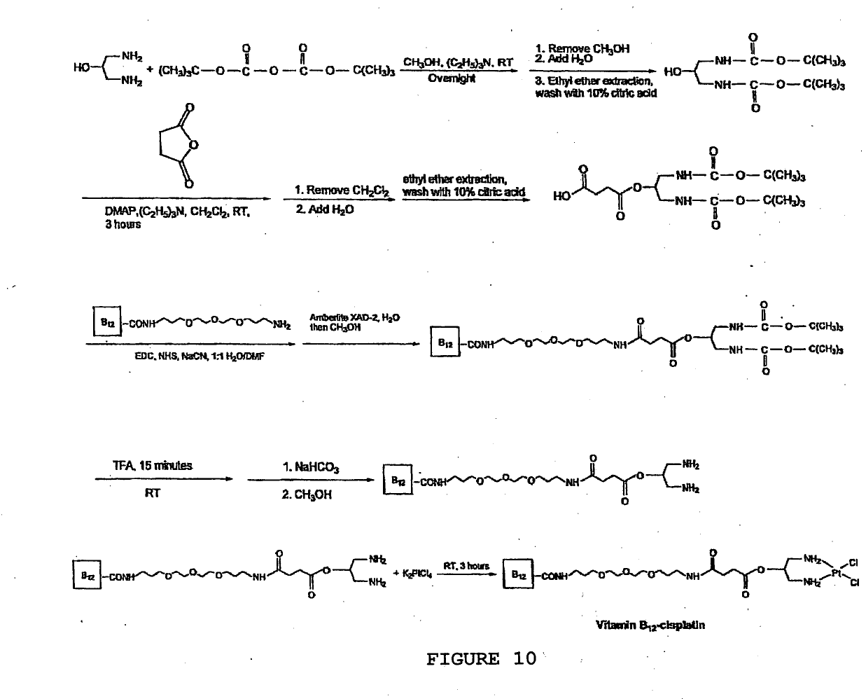

### DETAILED DESCRIPTION OF THE INVENTION

The present invention thus provides vitamin B₁₂ conjugates with anti-cancer drugs for targeted delivery of the anti-cancer drugs to tumor cells. This method works only in the presence of the vitamin B₁₂ carrier protein TCII, which is required for cell surface receptor binding and subsequent internalization.

The selectivity of the conjugates of the present invention for rapidly proliferating cells depends on the binding of the conjugate to TCII. For this reason, the e-linker is the preferred vitamin B₁₂ compound. Consequently, the conjugates of the present invention can be used for other diseases involving rapidly proliferating cells that require B₁₂, including rheumatoid arthritis, severe psoriasis, and neoplastic diseases.

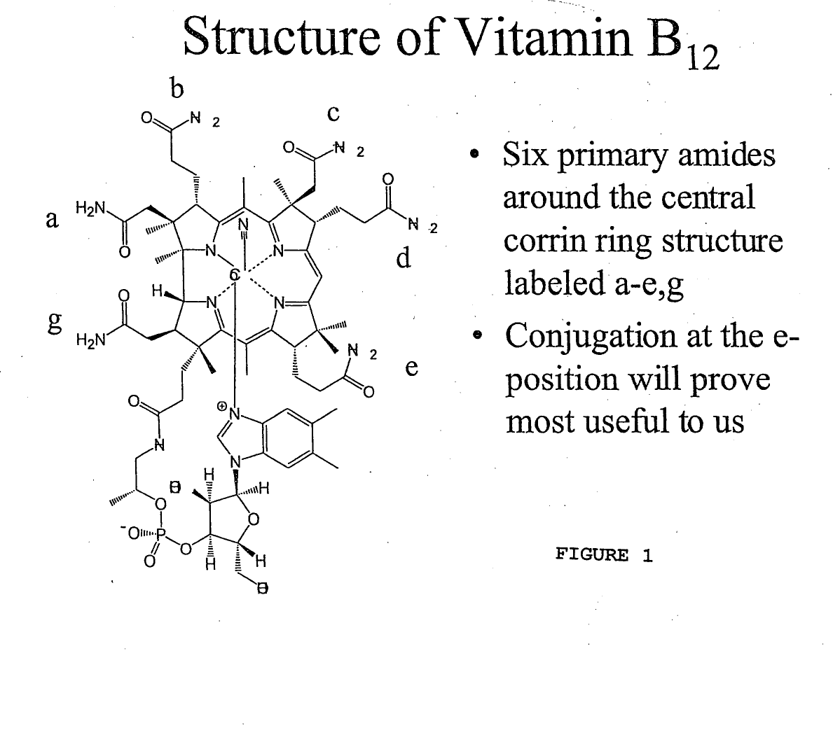

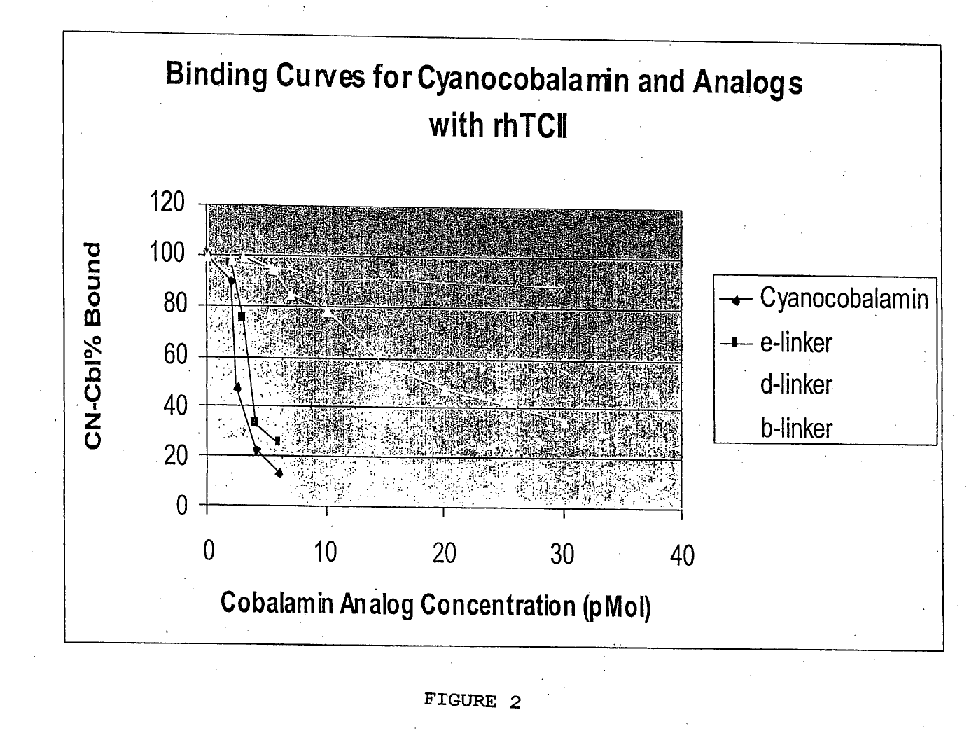

The structure of vitamin B₁₂ is shown in FIG. 1, in which the six primary amides are labeled a-e and g. Although the drugs can be conjugated at any of the six amide positions, it was found that conjugation at the e-position was most useful because vitamin B₁₂, cyanocobalamin, binds best with TCII when the drugs are conjugated at the e-amide position, as shown in FIG. 2.

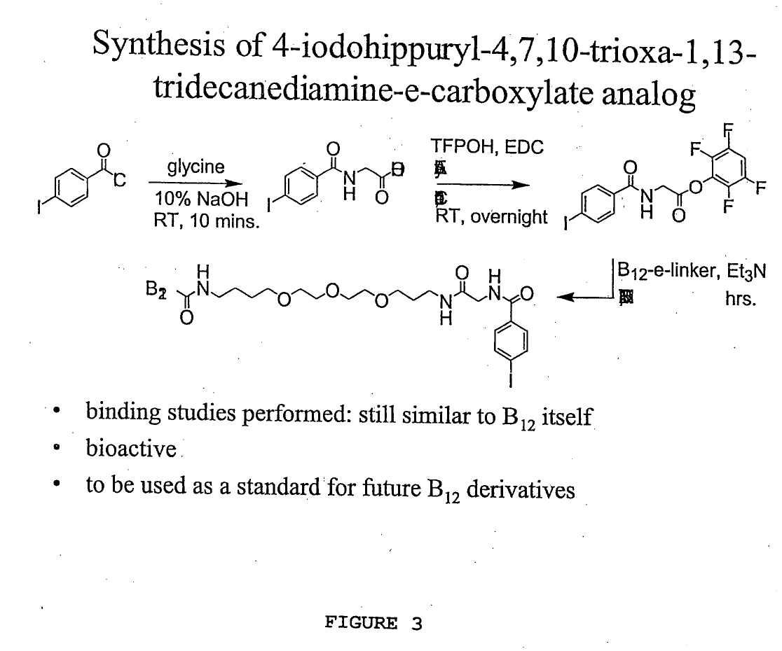

To illustrate this, 4-iodohippuryl-4,7,10-trioxa-1,13-tridecanediamine-3-carboxylate analog was prepared as shown in FIG. 3. In the binding studies performed, the conjugate was similar to vitamin B₁₂ alone, and was found to be bioactive. This conjugate may be used as a standard for future B₁₂ derivatives because of its higher rate of binding to TCII, which is required for efficient delivery to the appropriate receptors.

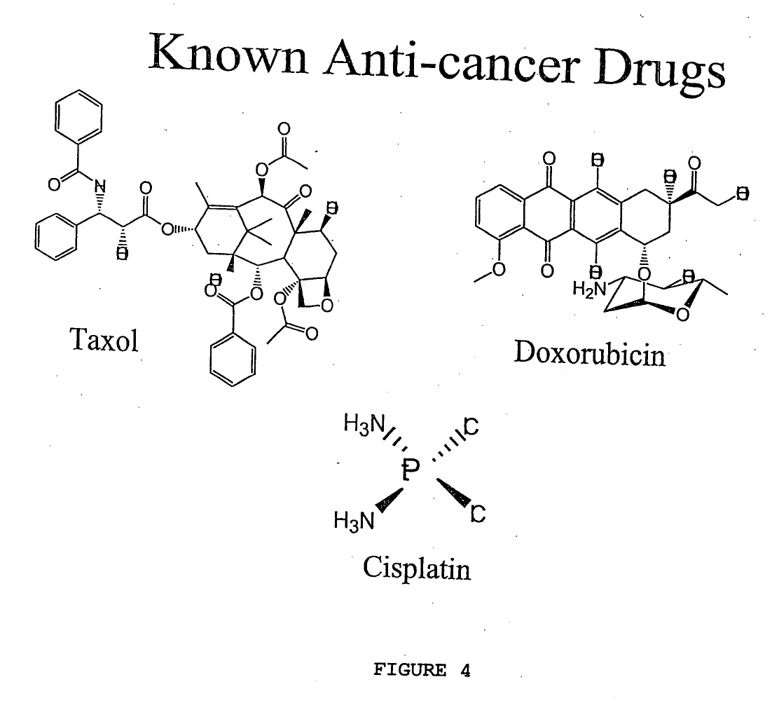

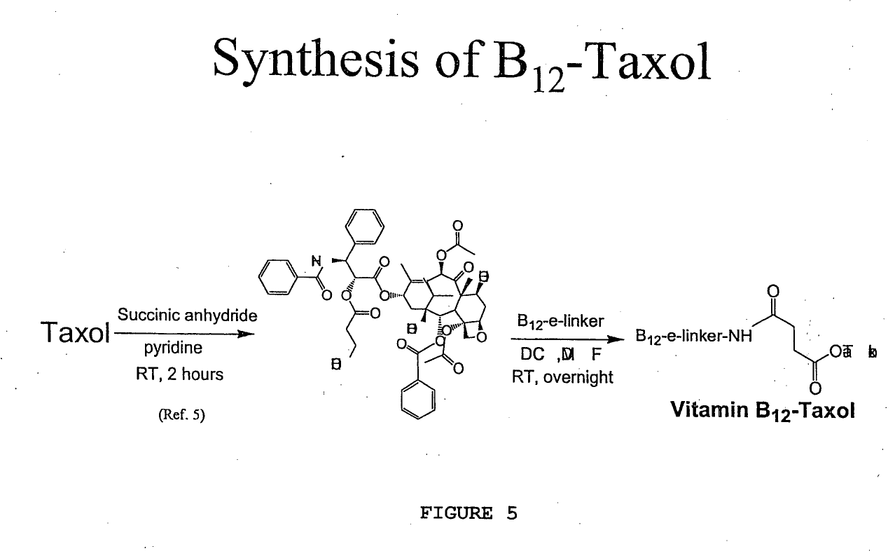

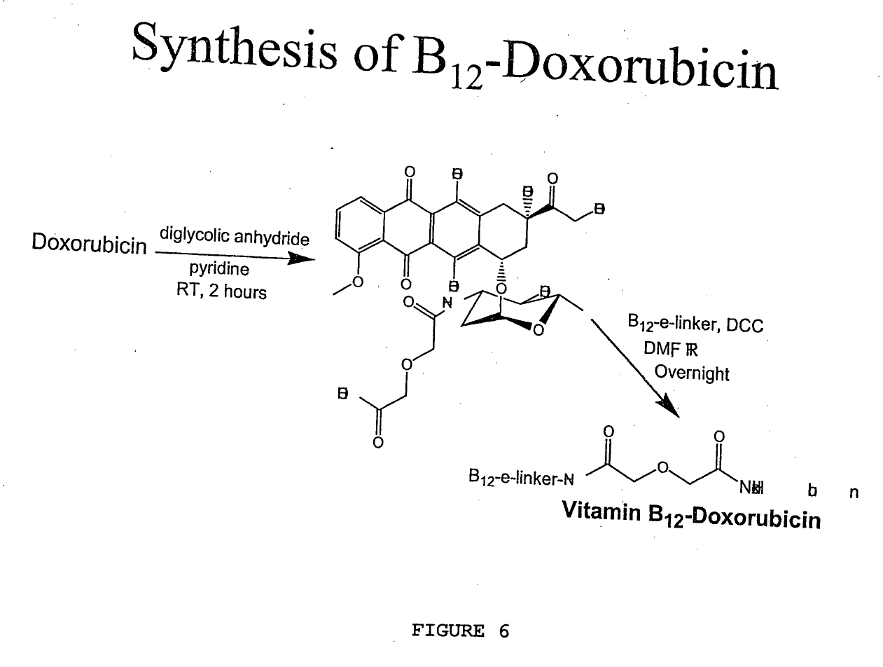

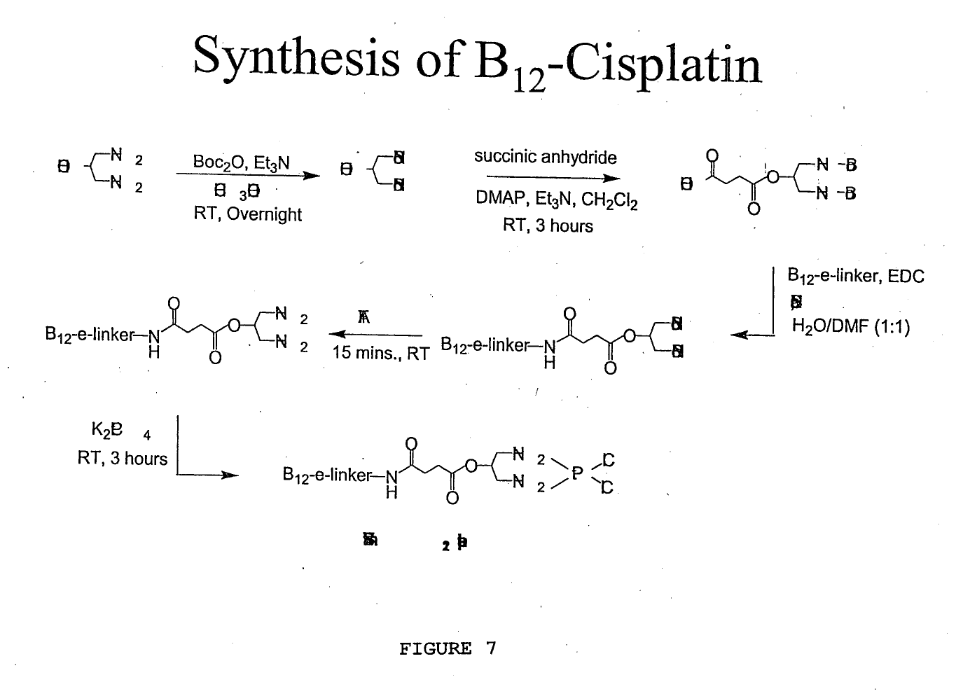

FIG. 4 illustrates three conventional anti-cancer drugs: taxol, doxorubicin, and cisplatin. Taxol and doxOrubicin contain an alcohol and an amino functionality, respectively, and therefore they must be derivatized to a carboxylic acid group before being compounded to the key vitamin B₁₂ unit, which contains an amino linker. As shown in FIG. 5, taxol was reacted with succinic anhydride to yield the carboxylic acid. This procedure was followed for doxorubicin using diglycolic anhydride rather than succinic anhydride, as shown in FIG. 6. Diglycolic anhydride was used for doxorubicin since the addition of an extra oxygen in the linker will enhance the overall water solubility of the final compound. Also, more than one product was obtained with doxOrubicin, since doxorubicin contains more than one nucleophilic substituent. However, the major product was a result of the reaction between the amine substituent and the anhydride. A C18 column was used to separate the two products. Both the taxol and the doxorubicin derivatized carboxylic acids were coupled to vitamin B₁₂ using the same procedure. FIG. 7 shows the synthesis of vitamin B₁₂-cisplatin.

Once a drug has been derivatized, if necessary, to provide a carboxylic acid to be linked to vitamin B₁₂, the same reaction sequence can be used to link any compound containing a nucleophilic functionality to vitamin B₁₂, as shown in Reaction Scheme 1.

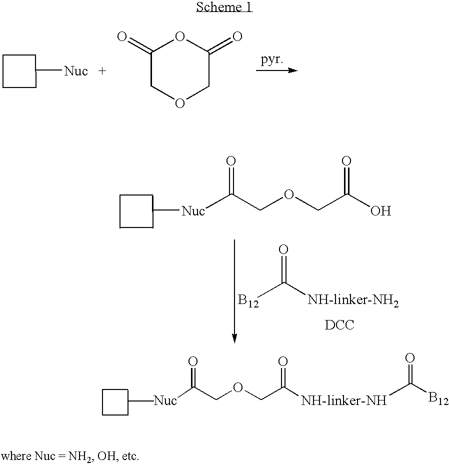

If the compound already contains a carboxylic acid, the underivatized compound can be coupled to the B₁₂ linker in one step, as shown in Scheme 2.

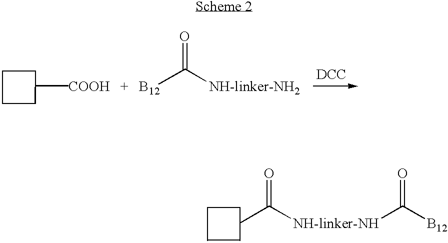

Additionally, cobalamin analogues according to the present invention interfere with HIV-1 integrase, one of the enzymes necessary for inserting the HIV virus into cellular DNA. Cobalamin analogues of the present invention can behave as antimetabolites by obstructing the normal usage of the cofactor. Consequently, vitamin B₁₂ can be used as a delivery vehicle by conjugating cobalamin and a therapeutic moiety.

In one embodiment of the present invention, three B₁₂ acid analogues, b, d, and e, were attached to a bioactive molecule, hippuric acid ester, via a twelve-carbon spacer molecule. It was found that spacer molecules having from 4 to 24 carbon atoms provide no hindrance to the binding of the analog to TCII. Longer >10 linkers are better although p-iodohippuryl-1,12-diaminododecane conjugated to cyanocobalamin-e-carboxylate was the most bioactive conjugate studied, the highly lipophilic diaminododecane made the compounds very insoluble in aqueous media and difficult to assay in biological systems. It was found that a more water-soluble linking moiety, 4,7,10-trioxa-1,13-tridecanediamine, made the conjugate more soluble in aqueous media and therefore more useful in treatment.

The cobalamin-linked drug molecules are transported into cancerous cells, where they remain biologically inert until the active drug is released from the covalent linker. One advantage of using cobalamin is that residual inactivated prodrug can be removed from the subjects following treatment. Cobalamin=s solubility in water allows it to be recovered from the urine by the kidneys and returned to the bloodstream through saturable receptors in the glomerulus.

Materials and Methods

Preparation of Cyanocobalamin Monocarboxylic Acids

Cyanocobalamin (3.7 mmol, 5 g) was dissolved in 500 mL of 0.1 N HCl. The mixture was stirred at room temperature of 10-11 days under argon. Because of cyanocobalamin=s sensitivity to light, the container was covered with aluminum foil. The solution was then neutralized with 6 N NaOH. The cobalamins were desalted by phenol extraction, after which the collected aqueous fractions were washed with 100 mL of 90% (w/w) phenol/water and twice with 25 mL and once with 10 mL of phenol. The phenol extracts were combined, and to this solution were added 200 mL water, 480 mL of diethyl ether, and 160 mL of acetone. To remove traces of phenol, the aqueous layer was isolated and washed with 30 mL ether.

The aqueous cobalamin solution was applied to a Dowex 1×2 column (200 g, 60×4 cm), which had been prepared by washing with saturated sodium acetate until it was free from CL⁻ a, and then washing with 200 mL water, acetate form, 200-400 mesh. The column was eluted with water to remove unreacted cyanocobalamin and then eluted with 0.04 M NaOAc, pH 4.7. The first fraction of the second elution contained three monocarboxylic acids. This fraction was desalted by phenol extraction as above. The aqueous solution of monocarboxylic acids was evaporated to dryness to yield 2.5 g (50%).

A 350 mg quantity of the mixture of three acids was then applied to 200 g of aminopropyl column packing (40-63 μm) in a glass column (1000 mm×25 mm) and was eluted with 58 μM pyridine acetate, pH 4.4 in H₂O/THF (96:4). The eluent was collected with an automatic fraction collector. The first eluted acid was found to be d-monocarboxylic acid; the second eluted acid was b-monocarboxylic acid; and the third eluted acid was e-monocarboxylic acid. The collected fractions were all checked by HPCL (Varian Star), and fractions containing pure samples were combined. The solids obtained were recrystallized from aqueous acetone to yield 560 mg (16%) of the d-isomer, 600 mg (17%) of the b-isomer, and 200 mg (5.7%) of the e-isomer.

Conjugation of Linker to Monocarboxylic Acids

[The cyanocobalamin monocarboxylic acids (b, d, and e), 500 mg, 0.370 mmol, and 170 mg, 148 mmol, N-hydroxysuccinimide (NHS) were dissolved in 18.4 mL of DMF/H2O (1:1), and the pH was adjusted to 6 with 1 N HCl. The 4,7,10-trioxa-1,13-tridecanediamine solution (EDC) was added in one portion to the cyanocobalamin solution. EDC (285 mg) was added, and the pH of the mixture was readjusted to 5.5. The reaction mixture was then stirred overnight in the dark at room temperature. In five intervals of 6-14 hours, 170 mg of NHS and 285 mg of EDC were added to the solution. The pH was adjusted to 5.5 each time. After a total reaction time of four days, monitored by HPLC, the solution was evaporated to dryness. The residue was digested with 100 mL of acetone, and the solvent was decanted. The solid residue was dissolved in 50 mL of H₂O and applied to an Amberlite XAD-2 column (200 g , 60×4 cm). The column was first washed with one liter of H₂O and then the desired product was eluted with 500 mL of methanol. The solution was evaporated to dryness, and the residue was dissolved in 25 mL of H₂O and was applied to a Dowex Cl⁻ (100 g, 60×25 cm, acetate form, 200-400 mesh). The product was eluted from the column with 250 mL of water, leaving any nonreacted acid bound to the column. This was followed by elution with 0.04 M sodium acetate buffer, pH 4.7. The fractions containing the final product were evaporated to dryness.

Synthesis of p-Iodohippuric Acid

A 5.3 g (7.1 mmol) quantity of glycine was dissolved in 100 mL of 10% NaOH. To this solution was added 19.4 g (7.3 mmol) of p-iodobenzoyl chloride in several portions. The reaction was allowed to stir for ten minutes and then was cooled in an ice-water bath. To facilitate stirring, 100 mL of water was added. The solution was then acidified to pH 5 with dropwise addition of 6 N HCl. The thick yellow faint precipitate was collected by vacuum filtration and dried. The crude product was recrystallized from boiling methanol to yield 15.3 g (70%) of off-white crystals, p-iodohippuric acid.

Synthesis of p-iodohippuric Acid TFP Ester

A solution containing 1.0 g (3.3 mmol) of p-iodohippuric acid suspended in 30 mL of anhydrous EtOAc was cooled in an ice-water bath. To this solution was added 1.6 g (9.6 mmol) of 2,3,5,6-tetrafluorophenol (TFR-OH) in 5 mL of anhydrous EtOAc, followed by 0.74 g (3.36 mmol) of EDC (1-[3-(dimethylamino)-propyl]-3-ethylcarbodiimide). The reaction was stirred for one hour at 0 EC and then allowed to warm to room temperature with stirring overnight. The EtOAc was removed by rotary evaporation to yield a tacky white solid. This material was dissolved in 10 mL of hexane, filtered, and dried to yield 0.89 g of a light yellow solid. The solid was eluted on a 2.5 cm×40 cm silica gel 60 column with a 25% EtOAc/1% HOAc/74% hexane eluent. Fractions of 100 mL were collected where the desired product eluted in fractions 6-11. These fractions were combined, and the solvent was removed to yield 0.79 g (53%) of the p-iodohippuric TFP ester as a white solid.

Preparation of p-iodohippuric acid-Cobalamin Conjugates

Acid hydrolysis of 5 g (3.7 mmol) cobalamin furnished 2.5 (50%) of crude monocarboxylic acid isomers (b, d and e). Chromatographic separation of the acid mixture resulted in 560 mg (16%) of the d-isomer, 600 mg (17%) of the b-isomer, and 200 mg (5.7%) of the e-isomer. Data from previous studies infer that the cyanocobalamin corrin ring e-carboxylate derivatives are most bioactive at very low concentrations, whereas derivatives prepared from conjugations at the d- and b-corrin ring carboxylate positions have varying bioactivities.

The p-iodohippuric acid TFP ester conjugated to the e-cyanocobalamin via the 4,7,10-trioxa-1,13-tridecanediamine linker was assessed for binding to transcobalamin II (TCII). It was found that the binding of the conjugate for TCII was substantially as great as for control vitamin B₁₂.

Paclitaxel-2-Succinate

Paclitaxel (20 mg, 0.234 mm) and 36 mg (0.3597 mmol) succinic anhydride were stirred in 0.06 mL pyridine at room temperature under nitrogen for three hours. A TLC was taken in 2:1 EtAc:hexane and showed that no starting material remained and one new spot had formed. The solvent was evaporated and the remaining yellowish solid was added to 1.5 mL H₂O and stirred for 20 minutes. The solid was filtered and then dissolved in acetone. Water was added, the solution was placed into an ice bath, and a white solid precipitated. The solid was filtered and dried. The yield was 12.8 mg, 57.4%, mp=167-169 EC (literature =178-180 EC).

B₁₂-Linker-Taxol

Paclitaxel-2-succinate (11.4 mg, 0.0119 mmol), 5 mg (0.0242 mmol) DCC and 0.1 mL DMF were stirred in a 1 mL microreactor for 30 minutes. Then e-B₁₂-trioxalinker-NH₂ (6.1 mg, 0.0039 mmol) in 0.3 mL DMF was added. This reaction was stirred overnight, covered with aluminum foil. An RP-C18 TLC was taken using 1:1 H₂O:MeOH. Two new red spots formed. The solvent was evaporated, and the reaction mixture was applied to a silica gel column eluting with 3:2 MeOH/CHCl₃ to remove all starting material. The fractions containing the two new red spots were then applied to an RP-C18 column eluting 3:1 H₂O/CH₃ CN to recover the desired product. The yield was 4.01 mg, or 41.3%).

Doxorubicin-COCH₂OCH₂COOH

Doxorubicin hydrochloride (100 mg, 0.173 mmol) and diglycolic anhydride (16.1 mg, 0.139 mmol) were stirred in 100 mL anhydrous pyridine in a microreactor vessel for 2.5 hours at room temperature. A reverse phase TLC was taken in 3:1 H₂O/CH₃CN, and a small amount of starting material remained. One new orange spot formed. The solvent was evaporated and the mixture was put onto an RP-C18 column to remove starting material. The product spot was eluted with 3:1H₂O/CH₃CN and the starting material remained in the column. The solvent was evaporated. The yield was 36.5 mg, or 40.1%.

B₁₂-Linker-Doxorubicin

Doxorubicin-COCH₂OCCH₂COOH (48 mg, 0.0728 mmol) and DCC (60.1 mg, 0.2912 mmol) were stirred in 10 mL DMF for 30 minutes. B₁₂-linker (28.3 mg, 0.182 mmol) was added and the mixture was stirred overnight, covered in aluminum foil. An RP-TLC was taken in 1:1 MeOH/H₂O with 1% ACOH. The solvent was evaporated and the mixture was applied to a C18 column; the product was eluted with 1:1 MeOH/H₂O with 1% ACOH. The starting material remained in the column. The yield of the product was 19.1 mg, or 47.4%. The product was dried.

Conjugation of e-isomer of Monocarboxylic Acid of Vitamin B₁₂ with 4,7,10-trioxa-1,13-tridecanediamine

In 100 mL H₂O, 880 mg e-isomer and 300 mg NHS(N-hydroxysuccinimide) were added. 640 mg NaCN was added to this mixture, followed by addition of 7.1 g 4,7,10-trioxa-1,13-tridecanediamine. The pH was adjusted to 6 with 1 N HCl. 510 mg of the 4,7,10-trioxaa-1,13-tridecanediamine solution (EDC) was added in one portion to the cyanocobalamin solution, and the pH of the mixture was readjusted to 5.5. The reaction mixture was then stirred overnight in the dark at room temperature. In five intervals of 6-14 hours, 300 mg of NHS and 510 mg of EDC were added to the solution. The pH was adjusted to 5.5 each time. After a total reaction time of four days, the solvent was removed under vacuum. The residue was then washed with 100 mL acetone. The solid residue was dissolved in 30 mL of H₂O and applied to an Amberlite XAD-2 column (200 g , 60×4 cm). The column was first washed with one liter of H₂O and then the desired product was eluted with 500 mL of methanol. The solution was evaporated to dryness, and the residue was dissolved in 25 mL of H₂O and was applied to a Dowex Cl⁻ (100 g, 60×25 cm, acetate form, 200-400 mesh). The product was eluted from the column with water, leaving any nonreacted acid bound to the column. This was followed by elution with 0.04 M sodium acetate buffer, pH 4.7 to elute the vitamin B₁₂ e-isomer. A total amount of 400 mg of conjugate was obtained. The yield was 39.6%.

Similar procedures were conducted for conjugating the b-isomer with 4,7,10-trioxa-1,13-tridecanediamine. Starting with 750 mg b-isomer, 546 mg. Product 2 was obtained, with a yield of 63.4%.

BOC Protection of Amino Groups of 1,3-Diamino-2-hydroxy Propane

In 30 mL methanol, 1.077 g of 1,3-dimino-2-hydroxy propane, 5.31 g di-tert-butyl dicarbonate, and 2.20 g triethanyl amine were added. The mixture was first stirred in a water-ice bath for one hour, and then was warmed up to room temperature and kept stirring overnight. The solvent was removed under vacuum. Then 30 mL CH₂Cl₂ was added to the residue and the organic layer was washed with 10% aqueous citric acid solution, 3×30 ml. The organic layer was dried with anhydrous magnesium sulfate before the solvent was removed under vacuum. The product was a colorless oil at room temperature. The weight of 2 was 2.279 g, with a yield of 70.5%.

Reaction between 2 and Succinic Anhydride

In 30 mL dry CH₂Cl₂, 2.270 g 2, 1.19 g triethylamine, and 0.964 g DMAP (4-dimethylaminopyridine) were added. The solution was maintained under stirring under nitrogen overnight. The color of the solution changed from colorless to brown-green after one hour, then to dark green overnight. The mixture was then washed with 10% citric acid aqueous solution, 5×40 mL. The organic layer was dried with anhydrous magnesium sulfate before the solvent was removed. The product 3 was a white solid. 1.61 g, or 49.4% yield, was isolated.

Conjugation between Vitamin B₁₂-linker 1 and 3

In 50 mL 1:1 v/v H₂O/DMF solution, 100 mg 1, 29.5 mg NHS, 62.9 mg NaCN and 247.9 mg 3 were added. The pH was adjusted to 6, then 49.2 mg EDC was added and the pH adjusted to 5.5. The solution was stirred under darkness. In five intervals between 6 and 14 hours, 29.5 mg NHS and 40.2 mg EDC were added. The reaction was kept going for four days. The reaction mixture was first purified by passing it through an Amberlite column, and further purification was by either ion exchange column or HPLC. This reaction is shown as Reaction Scheme 3. 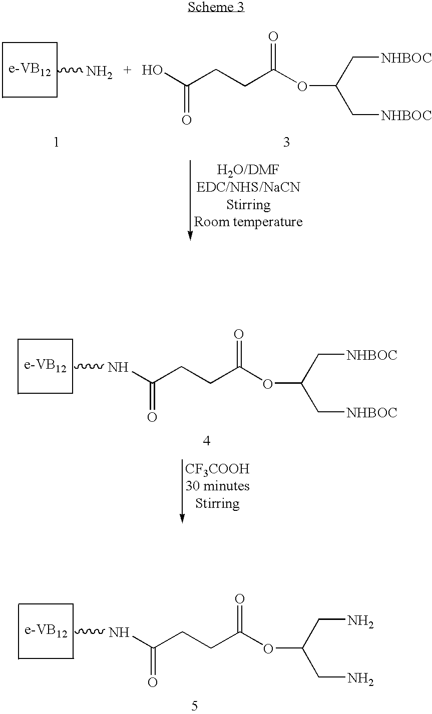
Experimental of Preparation of Vitamin B₁₂- Cisplatin Complex

1. Preparation of 4.^{1,2}

To 30 mL CH₃OH, 1.08 g 1,3-diamino-2-hydroxy propane and 5.31 g di-tert-butyl dicarbonate, and 2.20 g tri-ethyl amine were added. The mixture was kept stirring for 1 hour at 0 EC, then warmed up to room temperature and kept stirring overnight. The solvent was removed under vacuum. 30 ml CH₂Cl₂ was then added to the residue, then it was washed with 10% citric acid aqueous solution (2×30 mL). The organic layer was dried with anhydrous MgSO₄, CH₂Cl₂ was removed under vacuum. 2.28 g colorless oil product was obtained. The reaction is illustrated in Scheme 4.

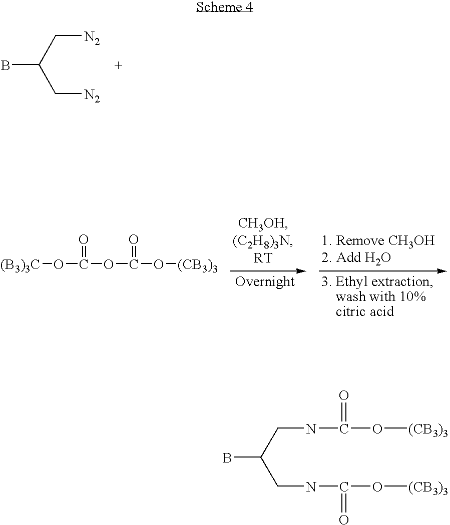

2. Preparation of 5^{3,4}

To 70 mL distilled CH₂Cl₂, 10 g 4, 7.7 g tri-ethyl amine, 9.3 g DMAP (4(Dimethylamino) pyridine) were added. The mixture was put in H₂O-ice bath and kept stirring. Slowly to the mixture, 7.6 g succinic anhydride was added. The solution was warmed to room temperature gradually. The reaction was allowed to proceed for 6 hours. The color changed from colorless to yellow, dark green, then finally to black finally. CH₃OH was added to terminate the reaction. The solvent was then removed under vacuum. The residue was dissolved in ether, then washed with 10% citric acid aqueous solution. The organic layer was dried and solvent was removed, 11.8 g white solid product was obtained. The reaction is illustrated in Scheme 5.

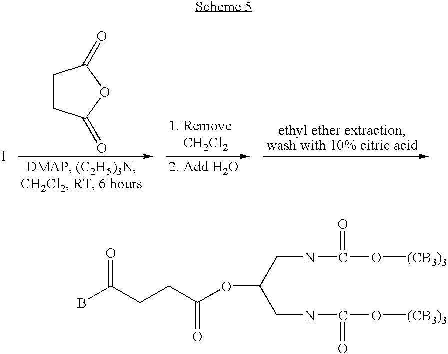

3. Preparation of 3.⁵

To 200 mL 1:1 v/v DMF/H₂O solution, add 720 mg B₁₂-linker, then 210 mg NHS, 470 mg NaCN, and 1.23 g 5. Adjust pH=6.Q using 6 M HCl. Then add 350 mg EDC, adjust pH=5.5. For the next five intervals between 8-16 hours, add 210 mg NHS and 350 mg EDC. Keep stirring at room temperature and in the dark. The solvent was removed in the dark. The solvent was removed under vacuum, then the mixture was desalted using XAD-2 column by H₂O eluting, the compound was then eluted by using methanol. The methanol was removed under vacuum, the residue was then dissolved in H₂O, then washed with ethyl ether to remove any remaining 5. The aqueous layer was then evaporated under vacuum to afford 600 mg 3. The reaction was illustrated in Scheme 6. 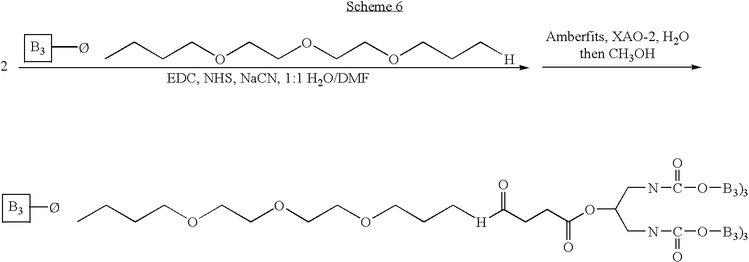
Preparation of B₁₂-cisplatin complex 7⁶

50 mg 6 was added to 2 mL TFA, stirred for 15 minutes to generate B₁₂ diamine. Then NaHCO₃ was added. The mixture was then put under vacuum to remove solvent. CH₃OH was added to the mixture, then filtered to obtain the B₁₂ diamine. Methanol was removed under vacuum. 5 mL H₂O'was added to the obtained B₁₂ diamine. 24 mg K₂PtCl₄ was dissolved in 1 mL H₂O, it was then added to the B₁₂ diamine mixture. The color changed from red to black-red after 30 minutes. The reaction was stopped after 3 hours, then the volume was reduced to about 1 mL. Acetone was then added to precipitate the product, 40 mg black-red product was obtained. The reaction was illustrated in Scheme 7.

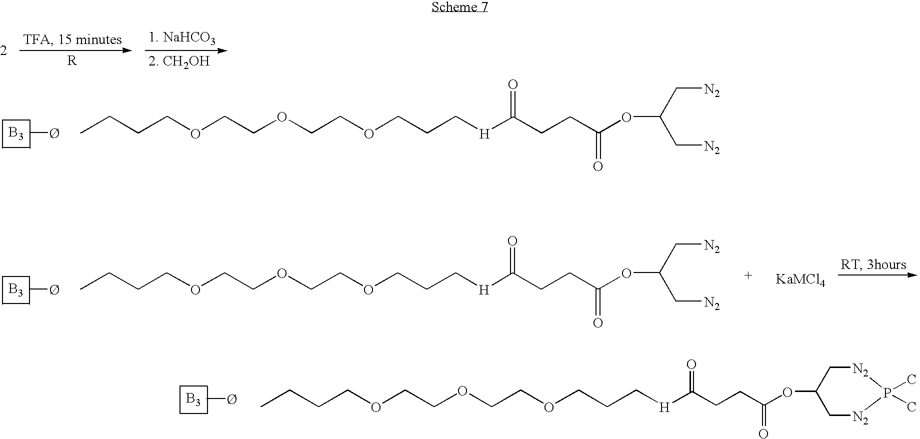

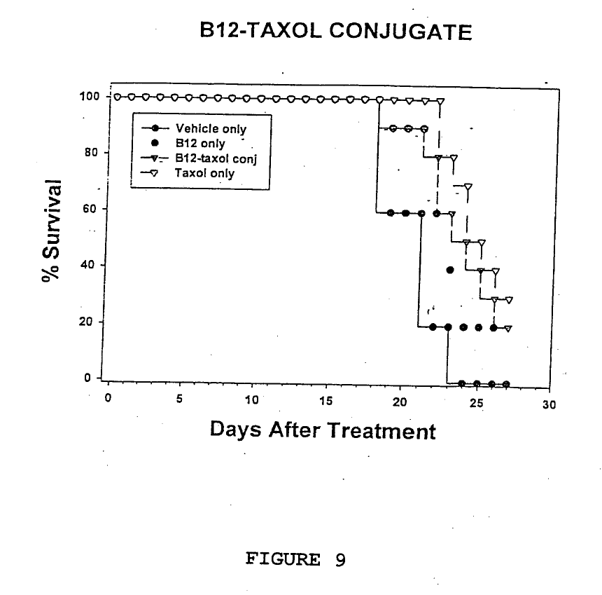

To determine the effects of a B₁₂-taxol conjugate on the leukemia lymphoma cell line P388, 30BDF1 male adult mice were given 0.1 ml intraperitoneal injections of 50,000 viable P388 cells each. 24 hours later the mice were then divided into four different groups and given single intraperitoneal injections of either vehicle alone, B₁₂ alone, taxol alone, or B₁₂-taxol conjugate. Daily observations were made and the total number of surviving mice in each group was recorded. The results are shown in FIG. 9.

The preparations used in the above experiment are as follows:

1. Vehicle alone: 1:5 (50:50 Cremophor:alcohol +5 parts 0.9% saline);

2. A single injection of 0.1 mL was administered intraperitoneally to each mouse;

3. B₁₂ alone: Vitamin B₁₂ dissolved in sterile water at a concentration of 26.4 mg/2 mL. A single dose of 66 mg/kg was given in a 0.1 mL intraperitoneal injection per mouse;

4. Taxol: 13.2 mg taxol dissolved in 0.6 mL (0.3 mL cremophor +0.3 mL ethanol), 2.4 mL of saline to give a concentration of 13.2 mg/3 mL. A single dose of 33 mg/kg was administered in a 0l:1 mL intraperitoneal injection per mouse;

5. B₁₂-taxol: B₁₂-taxol conjugate as described above was dissolved the same as taxol alone at a concentration of 50 mg/3 mL. A single dose of 99 mg/kg was given in a 0.1 mL intraperitoneal injection per mouse.

The cytotoxicity of B₁₂-taxol conjugate was tested against human cell lines grown in culture. The data below are means of duplicate experiments with similar results.

TABLE 1
Cytotoxicity of B₁₂-Taxol conjugate against
human cell lines grown in culture.

| | HL60 | | | |
|---|---|---|---|---|
| | Promyelocytic | | KARPAS-291 | K562 |
| | leukemia | | Lymphoma | Erythroleukemia |
| B₁₂ | 152 | ng/ml | 294 | 250 |
| Taxol | 0.15 | | 2.0 | 4.0 |
| B 12 (10 ng/ml) - | 0.14 | | 1.5 | 3.0 |
| Taxol | | | | |
| B₁₂-Taxol conjugate | 0.65 | | 1.5 | 6.4 |
| B₁₂-Taxol conjugate | 0.22 | | 0.51 | 2.2 |
| (as taxol | | | | |
| equivalents) | | | | |
| B₁₂ P-Hippuric | 245 | | 270 | 281 |
| acid* | | | | |

These three human cell lines were maintained in human serum (5%). B₁₂, taxol and the conjugate were prepared in a mixture of DMSO:PEG300 (1:1) for all studies. Cells were incubated with compounds continuously for 72 hr prior to assessment of relative cell growth by the MTT assay.
*B₁₂-P-Hippuric acid-4,7,10-trioxa-1,13-tridecanediamine e-cyanocobalamin monocarboxylic acid conjugate
Vitamin B₁₂ is essentially nontoxic against Karpas and K562 cell lines. Modest activity against HL60 was noted.
Taxol shows good activity against all three cell lines
The combination of taxol and B₁₂ (added as separate compounds to cells at the same time) shows a level of activity similar to that of taxol
The conjugate is also active against all three cell lines
Based on taxol-equivalents the conjugate was as active if not more active than taxol itself
The AB₁₂-hippuric acid@ conjugate appears to be no more active than B₁₂ itself

The conjugates of the present invention can be prepared with any known anticancer drug or drug to treat rapidly proliferating cells that require vitamin B₁₂. Among these drugs that can be conjugated to vitamin B₁₂ according to the present invention are azathioprine, aclacinomycin, aminoglutethinide, azathiprine, bicalutamide, bleomycin, bisulfan, camptothecin, carboplatin, carbofur, cefatamet pivoxil, ciprofloxacin, cisplatin, cladiribine, clomifene citrate, cyclophosphamide, cytarabine, cytarabine HCl, dacarbazine, dactinomycin, daunorubicin HCl, dequalinum chloride, docetaxel, doxifluridine, doxorubicin HCl, epirubicin, etoposide, famciclovir, fludarabine, fluoruracil, flutamide, foscarnet sodium, fosfamide, ftorafur, harringtonine, homogarringtonine, inobelbine, hydroxycamptothecin, hydroxycarbamide, hydroxyurea, idarubicin, ifosfamide, irinotecan, isotretinoin, leucovorin calcium, lumustine (CCNU), mercaptoprine, mesna, methotrexate, methotrexate disodium, mitomycin C, mitoxanthrone HCl, naftopidil, norcanthradidine, norcantharidiunum, ondasetron hydrochloride, oxaliplatin, penciclovir, ribavirin, rimantadine, stavudine, tamoxifen, base, tamoxifen, citrate, tegafur, topocetan, toremifenme citrate, ubenimex, valacyclovir, vancomycin, vinblastine sulfate, vincristine sulfate, and vindesine sulfate.

Pharmaceutical compositions according to the present invention can be administered by any convenient route, including parenteral, subcutaneous, intravenous, intramuscular, intra-peritoneal, or transdermal. Alternatively or concomitantly, administration may be by the oral route. The dosage administered depends upon the age, health, and weight of the recipient, nature of concurrent treatment, if any, and the nature of the effect desired.

Compositions within the scope of the present invention include all compositions wherein the active ingredient, i.e., conjugate, is contained in an amount effective to achieve its intended purpose. While individual needs vary, determination of optimal ranges of effective amounts of each compound is within the skill of the art. Typical dosages comprise 0.01 to 100 mg/kg body weight. The preferred dosages comprise 0.1 to 100 mg/kg body weight. The most preferred dosages comprise 1 to 50 mg/kg body weight.

Pharmaceutical compositions for administering the active ingredients of the present invention preferably contain, in addition to the conjugate, suitable pharmaceutically acceptable carriers comprising excipients and auxiliaries which facilitate processing of the active compounds into preparations which can be used pharmaceutically. Preferably, the preparations, particularly those preparations which are administered orally and which can be used for the preferred type of administration, such as tablets, dragees, and capsules, and also preparations which can be administered rectally, such as suppositories, as well as suitable solutions for administration by injection or orally, contain from about 0.01 to about 99 percent by weight, preferably from about 20 to 75 percent by weight, conjugate, together with the excipient. For purposes of the present invention, all percentages are by weight unless otherwise indicated. In addition to the following described pharmaceutical composition, the conjugates of the present invention can be formulated as inclusion complexes, such as cyclodextrin inclusion complexes.

The pharmaceutically acceptable carriers include vehicles, adjuvants, excipients, or diluents that are well known to those skilled in the art and which are readily available. It is preferred that the pharmaceutically acceptable carrier be one which is chemically inert to the conjugates and which has no detrimental side effects or toxicity under the conditions of use.

The choice of carrier is determined partly by the particular conjugate, as well as by the particular method used to administer the composition. Accordingly, there is a wide variety of suitable formulations of the pharmaceutical compositions of the present invention.

Formulations can be prepared for oral, aerosol, parenteral, subcutaneous, intravenous, intra arterial, intramuscular, intra peritoneal, intra tracheal, rectal, and vaginal administration.

Suitable excipients are, in particular, fillers such as saccharides, for example, lactose or sucrose, mannitol or sorbitol, cellulose preparations and/or calcium phosphates, for example, tricalcium phosphate or calcium hydrogen phosphate, as well as binders such as starch paste using, for example, maize starch, wheat starch, rice starch, potato starch, gelatin, tragacanth, methyl cellulose, hydroxypropylmethylcellulose, sodium catrboxymethylcelullose, and/or polyvinyl pyrrolidone.

Suitable formulations or parenteral administration include aqueous solutions of the conjugates in water-soluble form, such as when a hydrophilic linker is used. In addition, suspensions of the conjugate as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension, including, for example, sodium carboxymethyl cellulose, sorbitol, and/or dextran. Optionally, the suspension may also contain stabilizers.

Other pharmaceutically acceptable carries for the conjugates according to the present invention are liposomes, pharmaceutical compositions in which the active ingredient is contained either dispersed or variously present in corpuscles contained either dispersed or variously present in corpuscles consisting of aqueous concentric layers adherent to lipid layers. The active ingredient may be present both in the aqueous layer and in the lipidic layer, inside or outside, or, in any event, in the nonhomogeneous system generally known as a liposomic suspension.

The hydrophobic layer, or lipid layer, generally, but not exclusively, comprises phospholipids such as lecithin and sphingomyelin, steroids such as cholesterol, more or less ionic surface active substances such as dicetyl phosphate, stearylamine, or phosphatidic acid, and/or other materials of a hydrophobic nature.

The conjugates may also be formulated for transdermal administration, for example in the form of transdermal patches so as to achieve systemic administration.

Formulations suitable for oral administration can consists of liquid solutions such as effective amounts of the conjugates dissolved in diluents such as water, saline, or orange juice; capsules, tables, sachets, lozenges, and troches, each containing a predetermined amount of the active ingredient as solids or granules; powders, suspensions in an appropriate liquid; and suitable emulsions. Liquid formulations may include diluents such as water and alcohols, e.g., ethanol, benzyl alcohol, and the polyethylene alcohols, either with or without the addition of a pharmaceutically acceptable surfactant, suspending agents, or emulsifying agents. Capsule forms can be of the ordinary hard- or soft-shelled gelatin type containing, for example, surfactants, lubricant, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscaramellose sodium, talc, magnesium stearate, calcium stearate, zinc stearate, stearic acid, and other preservatives, flavoring agents, and pharmaceutically acceptable disintegrating agents, moistening agents preservatives flavoring agents, and pharmacologically compatible carriers. Lozenge forms can comprise the active ingredient in a carrier, usually sucrose and acacia or tragacanth, as well as pastilles comprising the active ingredient in an inert base such as gelatin or glycerin, or sucrose and acacia. Emulsions and the like can contain, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The conjugates can be administered in a physiologically acceptable diluent in a pharmaceutical carriers, such as a sterile liquid or mixture of liquids, including water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,3-dioxolane-4-methanol, ethers such as poly(ethylene glycol) 400, oils, fatty acids, fatty acid esters or glycerides, or acetylated fatty acid glycerides, without the addition of a pharmaceutically acceptable surfactants, such as soap or a detergent, suspending agent, such as carbomers, methylcellulose, hydroxypropylmethylcellulose, or carboxymethylcellulose, or emulsifying agents and other pharmaceutical adjuvants.

Oils which can be used in parenteral formulations include petroleum, animal, vegetable, or synthetic oils. Specific examples of oils include peanut, soybean, sesame, cottonseed, corn, olive, petrolatum, and mineral. Fatty acids can be used in parenteral formulations, including oleic acid, stearic acid, and isostearic acid. Ethyl oleate and isopropyl myristate are examples of suitable fatty acid esters. Suitable salts for use in parenteral formulations include fatty alkali metal, ammonium, and triethanolamine salts, and suitable detergents include cationic detergents such as dimethyl dialkyl ammonium halides, and alkyl pyridimium halides; anionic detergents such as dimethyl olefin sulfonates, alkyl, olefin, ether, and monoglyceride sulfates and sulfosuccinates; polyoxyethylenepolypropylene copolymers; amphoteric detergents such s alkyl-beta-aminopropionates and 2-alkyl-imidazoline quaternary ammonium salts; and mixtures thereof.

Parenteral formulations typically contain from about 0.5 to 25% by weight of the active ingredient in solution. Suitable preservatives and buffers can be used in these formulations. In order to minimize of eliminate irritation at the site of injection, these compositions may contain one or more nonionic surfactants having a hydrophilic-lipophlic balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be present in unit dose or multiple dose sealed containers, such as ampules and vials, and can be stored in a freeze-dried (lyophilized) condition requiring only the addition of the sterile liquid carrier, e.g., water, for injections immediately prior to use. Extemporaneous injection solutions and suspensions can be prepared from sterile powders, granules, and tablets of the kind previously described.

Additionally, the conjugates can be formulated into suppositories by mixing the active ingredient with a variety of bases, including emulsifying bases or water-soluble bases. Formulations suitable for vaginal administration may be in the form of pessaries, tampons, creams, gels, pastes, foam, or spray formulations containing, in addition to the active ingredient, such carriers as are known in the art to be appropriate.

The linking group can be chosen to provide water solubility or insolubility to the conjugates of the present invention. Thus, depending upon the linker used, the carrier Could include either an aqueous solution or a nonpolar liquid.

Any number of assays well known in the art may be used to test whether a particular conjugate functions as an anticancer drug, and one skilled in the art can readily determine if the conjugates of the present invention retain the properties of the original drug.

In determining the dosages to be administered, the dosage and frequency of administration is selected in relation to the pharmacological properties of the specific active ingredients. Normally, at least three dosage levels should be used. In toxicity studies in general, the highest dose should reach a toxic level but be sub lethal for most animals in the group. If possible, the lowest dose should induce a biologically demonstrable effect. These studies should be performed in parallel for each compound selected.

Additionally, the ID₅₀ level of the active ingredient in question can be one of the dosage levels selected, and the other two selected to reach a toxic level. The lowest dose that dose not exhibit a biologically demonstrable effect. The toxicology tests should be repeated using appropriate new doses calculated on the basis of the results obtained. Young, healthy mice or rats belonging to a well-defined strain are the first choice of species, an the first studies generally use the preferred route of administration. Control groups given a placebo or which are untreated are included in the tests. Tests for general toxicity, as outlined above, should normally be repeated in another non-rodent species, e.g., a rabbit or dog. Studies may also be repeated using alternate routes of administration.

Single dose toxicity tests should be conducted in such a way that signs of acute toxicity are revealed and the mode of death determined. The dosage to be administered is calculated on the basis of the results obtained in the above-mentioned toxicity tests. It may be desired not to continue studying all of the initially selected conjugates. Data on single dose toxicity, e.g., LD₅₀, the dosage at which half of the experimental animals die, is to be expressed in units of weight or volume per kg of body weight and should generally be furnished for at least two species with different modes of administration. In addition to the ID₅₀ value in rodents, it is desirable to determine the highest tolerated dose and/or lowest lethal dose for other species, i.e., dog and rabbit.

When a suitable and presumably safe dosage level has been established as outlined above, studies on the drugs chronic toxicity, its effect on reproduction, and potential mutagenicity may also be required in order to ensure that the calculated appropriate dosage range will be safe, also with regard to these hazards.

Pharmacological animal studies on pharmacokinetics revealing, e.g., absorption, distribution, biotransformation, and excretion of the active ingredient and metabolites are then performed. Using the results obtained, studies on human pharmacology are then designed. Studies of the pharmacodynamics and pharmacokinetics of the compounds in humans should be performed in healthy subjects using the routes of administration intended for clinical use, and can be repeated in patients. The dose-response relationship when different doses are given, or when several types of conjugates or combinations of conjugates and free compounds are given, should be studied in order to elucidate the dose-response relationship (dose vs. plasma concentration vs. effect), the therapeutic range, and the optimum dose interval. Also, studies on time-effect relationship, e.g., studies into the time-course of the effect and studies on different organs in order to elucidate the desired and undesired pharmacological effects of the drug, in particular on other vital organ systems, should be performed.

The conjugates of the present invention are then ready for clinical trials to compare the efficacy of the conjugates to existing therapy. A dose-response relationship to therapeutic effect and for side effects can be more finely established at this point.

The amount of conjugates of the present invention to be administered to any given patient must be determined empirically, and will differ depending upon the condition of the patients. Relatively small amounts of the conjugate can be administered at first, with steadily increasing dosages if no adverse effects are noted. Of course, the maximum safe toxicity dosage as determined in routine animal toxicity studies.

The foregoing description of the specific embodiments will so fully reveal the general nature of the invention that others can, by applying current knowledge, readily modify and/or adapt for various applications such specific embodiments without departing from the generic concept, and, therefore, such adaptions and modifications should and are intended to be comprehended within the meaning and range of equivalents of the disclosed embodiments. It is to be understood that the phraseology or terminology employed herein is for the purpose of description and not of limitation.

### REFERENCES

Yuste, F.; Ortiz B, Carrasco A.; Peralta M.; Quintero L., Sanchez-Ohregon R.; Walls F.; Ruano J. L. G. Tetraheciron: Asymmety 2000, 11, 3079.
Basel, Y. Hassner A. J. Org. Chem 2000, 65, 6368.
Steglich, W.; Höfle, G. Angew. Chem. 1969, 81, 1001.
Höfle, G.; Steglich, W. Synthesis 1972, 619
Pathare, P. M.; Wilbur, D. S.; Heusser, S.; Quadros, E. V.; McLoughlin, P.; Morgan, A. C. Bioconjugate Chem. 1996, 7, 217.
Schütte, M. T.; Mülhaupt, R. Kratz, F. Metal-based Drugs 2000, 7, 89.

What is claimed is:

1. A method for treating a patient suffering from cancer comprising administering to said patient an effective amount of a conjugate of vitamin B₁₂ and an anti-cancer drug in the presence of transcobalamin II, which conjugate binds to transcobalamin II.

2. The method according to claim 1 wherein the anti-cancer drug is selected from the group consisting of taxol, doxorubicin, and cisplatin.

3. The method according to claim 1 wherein the vitamin B₁₂ is the e-isomer.

4. The method according to claim 1 wherein the spacer is selected from the group consisting of compounds having a —(CH₂)ₙ group, wherein n is from 4 to 20.

5. The method according to claim 4, wherein the linker is 4,7,10-trioxa-1,13-tridecanediamine.

6. A method for delivering drugs to rapidly dividing cells comprising administering to a patient in need thereof an effective amount of a conjugate of vitamin B₁₂ and a drug for treating rapidly dividing cells in the presence of transcobalamin II, which conjugate binds to transcobalamin II.

7. The method according to claim 6 wherein the rapidly dividing cells indicate a disease selected from the group consisting of rheumatoid arthritis, severe psoriasis, and neoplastic diseases.

8. The method according to claim 6 wherein the vitamin B₁₂ is the e-isomer.

9. The method according to claim 6 wherein the linker is selected from compounds having the group —(CH₂)ₙ wherein n is from 4 to 20.

10. The method according to claim 9 wherein the linker is 4,7,10-trioxa-1,3,-tridecanediamine.
